(19) United States
(12) Patent Application Publication
(10) Pub. No.: US 2005/0107690 A1
(43) Pub. Date: May 19, 2005

(54) Medical image interpretation system and interpretation report generating method

(75) Inventor: Shusuke Soejima, Tokyo (JP)

Correspondence Address: Finnegan, Henderson, Farabow,;Garrett & Dunner, L.L.P., 1300 I Street, N.W., Washington, DC, 20005-3315, US

(73) Assignee: KONICA MINOLTA MEDICAL & GRAPHIC, INC.

(21) Appl. No.: 10/986,191

(22) Filed: Nov. 12, 2004

(30) Foreign Application Priority Data
Nov. 13, 2003 (JP) 2003-383587

(51) Int. Cl. A61B 5/05

(52) U.S. Cl. 600/425

(57) ABSTRACT

A medical image interpretation system for displaying a medical image generated from a subject and for generating an interpretation report of the medical image, includes: a drawing section for drawing an annotation on an area of interest over the displayed medical image, the annotation being previously related to a text for interpretation report generation; and a report generating section for obtaining drawn position information and drawn size information of the annotation drawn by the drawing section, for obtaining region information of the medical image, and for generating the interpretation report of the medical image based on the obtained information and the text for interpretation report generation corresponding to the drawn annotation.

### BACKGROUND OF THE INVENTION

1. Field of the Invention

The present invention relates to a medical image interpretation system and an interpretation report generating method for interpreting a medical image and for generating a report.

2. Description of Related Art

These days, a medical image interpretation system is being used. To such a medical image interpretation system, connected are an information management apparatus for storing and managing a medical image, an interpretation terminal for displaying a medical image on a CRT (Cathode Ray Tube) or the like, and various types of image recording modalities for radiographing (generating an image of) a subject to generate image data of a medical image, such as a CT (Computed Tomography), an MRI (Magnetic Resonance Imaging), a CR (Computed Radiography), an FPD (Flat Panel Detector), a sonography apparatus and the like, through a network.

In this medical image interpretation system, a doctor who interprets a medical image (hereinafter, it is called an interpretation doctor) has a medical image displayed on the interpretation terminal for performing the interpretation, creates an interpretation report according to the findings (comments) and creates a diagnosis report from the diagnosis result. In order to make it easier to create (generate) these reports, various technologies have been proposed. For example, a technology which reads in diagnosis information which is written with a handwriting input pen on diagnosis report paper placed on a tablet as contact location data, and converts the read contact location data into a character code as handwriting information to generate an electronic file with image data is disclosed (see JP-Tokukaihei-11-31187A, hereinafter, it is called “document 1”).

In the above-described document 1, since it is not necessary to do the input, the kanji conversion and the like with a keyboard operation, it is possible to provide a user-friendly system to an interpretation doctor who is not accustomed to a keyboard operation. However, if this technology is applied to a case of generating an interpretation report, it is necessary to have a labor for converting the interpretation result into a text to be inputted. Further, if each interpretation doctor converts the interpretation result of a medical image into a text to be inputted in a conventional way, a text structure of the interpretation report takes various styles, and thereby, it is not possible to unify the text structure in a hospital.

### SUMMARY OF THE INVENTION

An object of the present invention is to provide a medical image interpretation system and an interpretation report generating method which are capable of easily generating an interpretation report of a medical image and of unifying a text structure of the interpretation report in a hospital.

In accordance with a first aspect of the present invention, a medical image interpretation system for displaying a medical image generated from a subject and for generating an interpretation report of the medical image, comprises: a drawing section for drawing an annotation on an area of interest over the displayed medical image, the annotation being previously related to a text for interpretation report generation; and a report generating section for obtaining drawn position information and drawn size information of the annotation drawn by the drawing section, for obtaining region information of the medical image, and for generating the interpretation report of the medical image based on the obtained information and the text for interpretation report generation corresponding to the drawn annotation.

According to the system of the first aspect, when an annotation with which a text for report generation is previously related is drawn over an area of interest on a displayed medical image, drawn position information and drawn size information of the drawn annotation as well as region information of the medical image are obtained. Then, based on these obtained information and a text for interpretation report generation corresponding to the drawn annotation, an interpretation report of the medical image is generated. Therefore, it is not necessary to have a doctor create a text of an interpretation report and input it with a keyboard or the like, whereby it is possible to easily generate an interpretation report. Further, since an interpretation report is automatically generated based on a text corresponding to the drawn annotation, it is possible to unify a text structure of interpretation reports in a hospital.

Preferably, the system of the first aspect of the present invention further comprises a storing section for storing image data of the medical image and image report data generated by drawing the annotation so as to relate each other.

According to the above-mentioned system, image data of a medical image and image report data which is generated by drawing an annotation over the medical image are stored so as to relate each other. Therefore, it is possible to utilize the image report data generated by drawing an annotation over the medical image in various ways.

Preferably, the system of the first aspect of the present invention further comprises a comparison report generating section for obtaining image report data of a previous image which is generated according to an identical region of an identical subject to the medical image, and for generating a comparison report by comparing the image report data of the medical image with the image report data of the previous image.

According to the above-mentioned system, by obtaining image data of a previous image in which an identical region of an identical human body to the medical image is radiographed (image-generated) from the storing section, it is possible to generate a comparison report by comparing image report data of the medical image with image report data of its previous image.

In accordance with a second aspect of the present invention, an interpretation report generating method in a medical image interpretation system for displaying a medical image generated from a subject and for generating an interpretation report of the medical image, comprises: drawing an annotation on an area of interest over the displayed medical image, the annotation being previously related to a text for interpretation report generation; obtaining drawn position information and drawn size information of the drawn annotation; obtaining region information of the medical image; and generating the interpretation report of the medical image based on the obtained information and the text for interpretation report generation corresponding to the drawn annotation.

According to the method of the second aspect, when an annotation with which a text for report generation is previously related is drawn over an area of interest on a displayed medical image, drawn position information and drawn size information of the drawn annotation as well as region information of the medical image are obtained. Then, based on these obtained information and a text for interpretation report generation corresponding to the drawn annotation, an interpretation report of the medical image is generated. Therefore, it is not necessary to have a doctor create a text of an interpretation report and input it with a keyboard or the like, whereby it is possible to easily generate an interpretation report. Further, since an interpretation report is automatically generated based on a text corresponding to the drawn annotation, it is possible to unify a text structure of interpretation reports in a hospital.

### BRIEF DESCRIPTION OF THE DRAWINGS

The present invention will become more fully understood from the detailed description given hereinafter and the accompanying drawing given by way of illustration only, and thus are not intended as a definition of the limits of the present invention, and wherein:

FIG. 1 is a view showing a whole structure of a medical image interpretation system 100 according to the present invention,

FIG. 2 is a block diagram showing a functional structure of an information management apparatus 2 of FIG. 1,

FIG. 3 is a view showing a data storing example of an image report DB 252 stored in a storing unit 25 of FIG. 2,

FIG. 4 is a block diagram showing a functional structure of an interpretation terminal 3 of FIG. 1,

FIG. 5 is a view showing a data storing example of an annotation storing unit 351 stored in a storing unit 35 of FIG. 4,

FIG. 6 is a flowchart illustrating an image report generating process executed by a CPU 31 of FIG. 4,

FIG. 7 is a view showing one example of an interpretation screen 331 displayed on a displaying unit 33 of FIG. 4,

FIG. 8 is a view showing an example of generating a report on the interpretation screen 331 of FIG. 7 on which annotation is drawn, and

FIG. 9 is a flowchart illustrating a comparison report generating process executed by the CPU 31 of FIG. 4.

### PREFERRED EMBODIMENTS OF THE INVENTION

Hereinafter, an embodiment of the present invention will be described with reference to figures. However, the scope of the invention is not limited to the illustrated examples.

First, a structure of the present embodiment will be described.

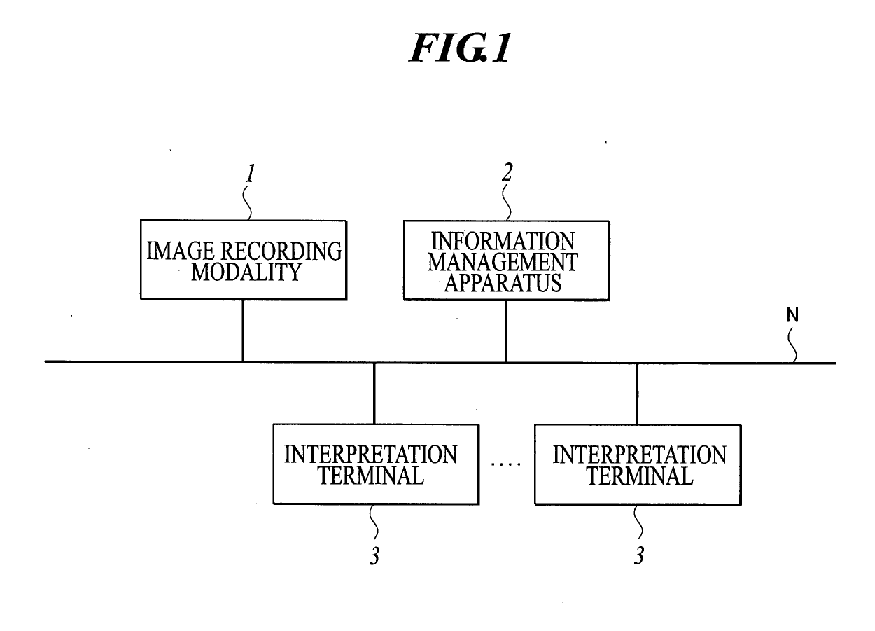

FIG. 1 is a conceptual view showing a system structure of a medical image interpretation system 100 in the present embodiment. As shown in FIG. 1, the medical image interpretation system 100 comprises an image recording modality 1, an information management apparatus 2, an interpretation terminal 3 and the like, and they are connected to each other so that data can be transmitted/received with each other through a network N. The number of the image recording modality 1 and the number of the interpretation terminal 3 are not in particular limited.

To the network N, various types of forms can be applied, such as LAN (Local Area Network), WAN (Wide Area Network), Internet and the like. In addition, if it is permitted to use the network N in a medical facility such as a hospital, the network can be a wireless communication or an infrared communication. Here, since it includes important patient information, information to be transmitted/received is preferably encoded.

The image recording modality 1 is an apparatus for recording a medical image in which an image of a subject (human body) is generated as digital data, such as CT, MRI, CR, FPD, sonography apparatus and the like. This apparatus may be one which digitally converts analog image signals obtained from radiography (image generation) and records the converted data as it is, or may be one which reads a medical image recorded temporarily in a film at radiography (image generation) for obtaining digital data. In the present embodiment, the image recording modality 1 requests the information management apparatus 2 to store the recorded image data therein. Here, it is assumed that image accompanying information (hereafter, it is called accompanying information) is added to the image data which is transmitted to the information management apparatus 2.

What can be cited as the accompanying information is, patient information regarding a patient such as a patient name of a patient to be radiographed (image-generated), patient ID, age, sex and the like; examination information such as examination date, examination ID, part information, radiographing (image-generating) condition (body position, radiographing (image-generating) direction and the like), image recording modality information and the like; image data information such as pixel number of a medical image, bit number, designated output size, reading pixel size, maximum density and the like; etc.

The information management apparatus 2 stores image data of the medical image recorded by the image recording modality 1 in an image DB (Data Base) 251 for a management purpose so as to relate it with its accompanying information, and supplies the image data to the interpretation terminal 3 according to a request from the interpretation terminal 3. Further, the information management apparatus 2 stores and manages image report data generated in an image report generating process (which will be described later) in the interpretation terminal 3, and supplies the image report data according to a request from the interpretation terminal 3.

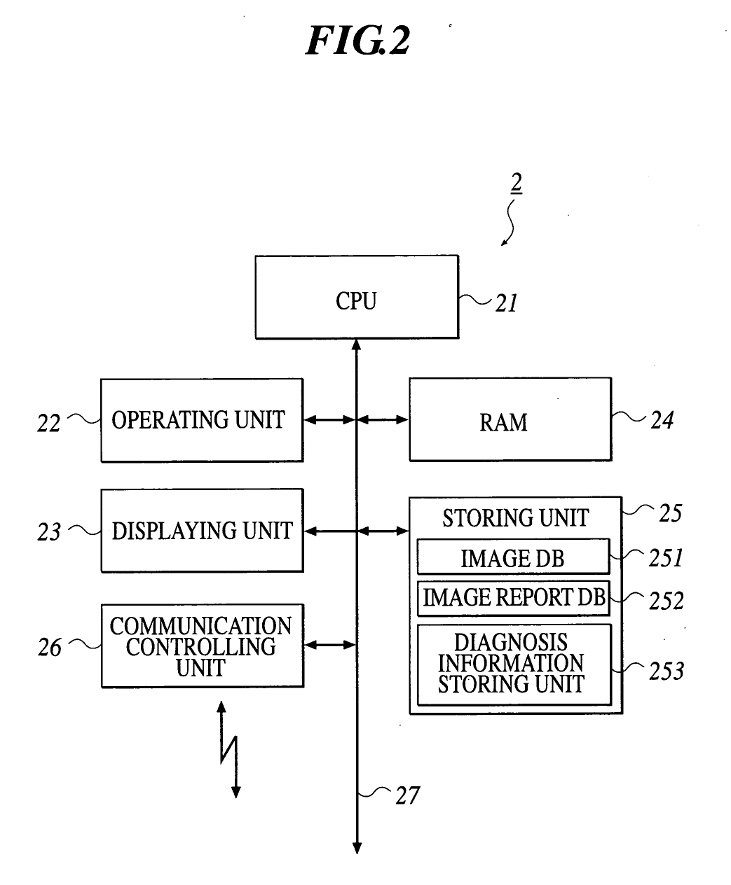

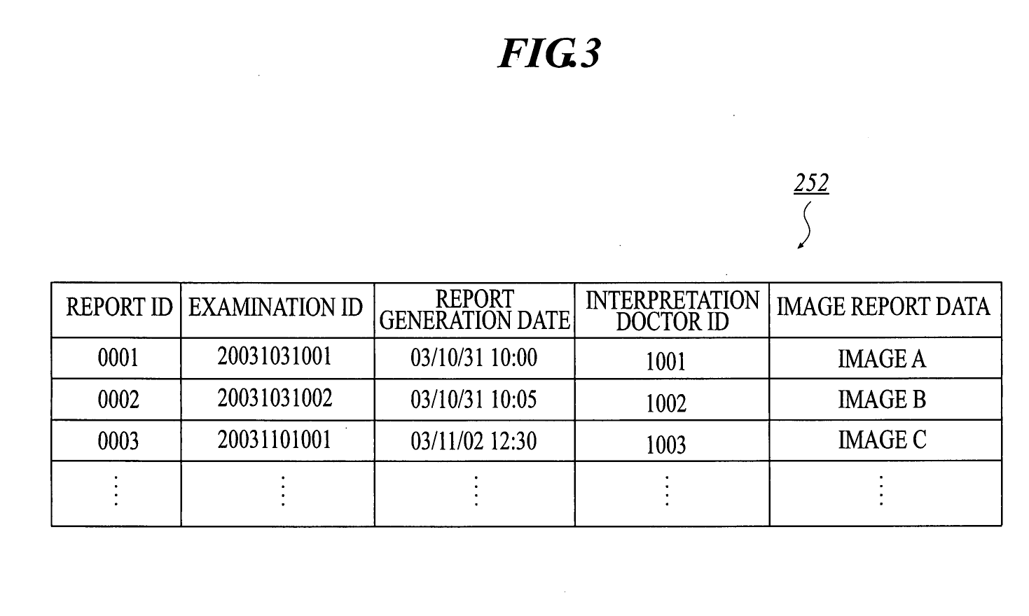

Here, with reference to FIG. 2, an internal structure of the information management apparatus 2 will be described.

FIG. 2 is a block diagram showing a functional structure of the information management apparatus 2. As shown in FIG. 2, the information management apparatus 2 comprises a CPU 21, an operating unit 22, a displaying unit 23, a RAM 24, a storing unit 25, a communication controlling unit 26 and the like, and each unit is connected through a bus 27.

The CPU 21 reads out a system program stored in the storing unit 25, develops the system program into a work area which is formed in the RAM 24, and controls each unit according to the system program. Further, the CPU 21 reads out various types of processing programs and various types of application programs stored in the storing unit 25, and develops them into the work area to execute various types of processes.

The operating unit 22 comprises a keyboard having cursor keys, numeric keys, various function keys and the like, and a pointing device such as a mouse or the like, and outputs a instruction signal inputted according to a key operation on the keyboard or a mouse operation to the CPU 21. Further, the operating unit 22 may be realized with a touch panel provided on the display screen of the displaying unit 23. In this case, an instruction signal inputted through the touch panel is outputted to the CPU 21.

The displaying unit 23 comprises a monitor such as LCD (Liquid Crystal Display), CRT or the like. The displaying unit 23 displays an input instruction from the operating unit 22, data and the like according to an instruction of a display signal inputted from the CPU 21.

The RAM 24 forms a storing area for temporarily storing various programs read out from the storing unit 25, the various programs being executable by the CPU 21, input data, output data, a parameter and the like, in various processes whose execution is controlled by the CPU 21.

The storing unit 25 comprises HDD (Hard Disc Drive), nonvolatile semiconductor memory or the like, and stores the system program executed by the CPU 21, various processing programs corresponding to the system program, various application programs, various data and the like. These various programs are stored as readable program codes, and the CPU 21 sequentially executes an operation according to the program codes.

Further, as shown in FIG. 2, the storing unit 25 comprises an image DB (Data Base) 251, an image report DB 252 and a diagnosis information storing unit 253.

The image DB 251 stores image data of a medical image received from the image recording modality 1 so as to relate it with accompanying information of the image data. The accompanying information is, for example, stored in a header of each image data.

As the storing section, the image report DB 252 is a database for storing image report data generated by the interpretation terminal 3 and information regarding the image report data. For example, the image report DB 252 stores a report ID being an identification code for uniquely identifying the image report data (for example, 0001, 0002, 0003, . . . ), an examination ID for identifying image data used for generating the image report data (for example, 20031031001, 20031031002, 20031101001, . . . ), date and time data which indicates a generation date and time of generating the image report data (for example, 03/10/31 10:00, 03/10/31 10:15, 03/11/02 12:30, . . . ), an interpretation doctor ID being an identification code for identifying an interpretation doctor who creates the image report data (for example, 1001, 1002, 1003), and the image report data (image A, image B, image C, . . . (each of images A to C is image data of a medical image on which annotation (a figure for indicating an area of interest, such as oval, rectangle or the like) is added)) the image data being related according to the examination ID, so as to relate each other. Each record in the image report DB 252 is related to the image data and its accompanying information in the image DB 251, according to the examination ID.

The diagnosis information storing unit 253 stores diagnosis information generated by the interpretation terminal 3 so as to relate it with the image data in the image DB 251 according to the examination ID.

The communication controlling unit 26 comprises a LAN adapter, a router, TA (Terminal Adapter) or the like, and controls communications among each apparatus connected to the network N.

The interpretation terminal 3 is a terminal such as PC (Personal Computer) or the like, which is set up for a purpose of displaying a medical image and generating an interpretation report of the medical image (hereafter, it is called a report).

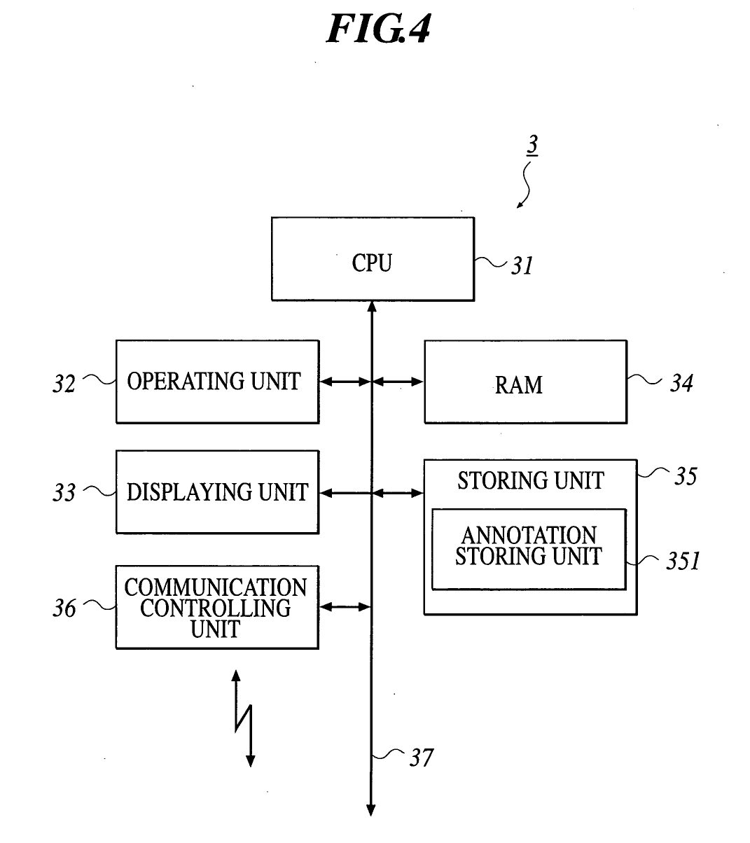

Here, with reference to FIG. 4, an internal structure of the interpretation terminal 3 will be described.

FIG. 4 is a block diagram showing a functional structure of the interpretation terminal 3. As shown in FIG. 4, the interpretation terminal 3 comprises a CPU 31, an operating unit 32, a displaying unit 33, a RAM 34, a storing unit 35, a communication controlling unit 36 and the like, and each unit is connected through a bus 37.

The CPU 31 reads out a system program stored in the storing unit 35, develops the system program into a work area formed in the RAM 34 and controls each unit according to the system program. Further, the CPU 31 reads out various processing programs and various application programs stored in the storing unit 35, and develops them into the work area to execute various processes such as an image report generating process, a comparison report generating process (each will be described later) and the like.

The operating unit 32 comprises a keyboard having cursor keys, numeric keys, various function keys and the like, and a pointing device such as a mouse or the like, and outputs an instruction signal inputted according to a key operation on the keyboard or a mouse operation to the CPU 31. Further, the operating unit 32 may be realized by providing a touch panel on the display screen of the displaying unit 33. In this case, a instruction signal inputted through the touch panel is outputted to the CPU 31.

The displaying unit 33 comprises a monitor such as an LCD (Liquid Crystal Display), a CRT or the like. The displaying unit 33 displays an input instruction from the operating unit 32, data and the like according to an instruction of a display signal inputted from the CPU 31.

The RAM 34 forms a storing area for temporarily storing various programs read out from the storing unit 35, the various programs being executable by the CPU 31, input data, output data, a parameter and the like, in various processes whose execution is controlled by the CPU 31.

The storing unit 35 comprises an HDD (Hard Disc Drive), a nonvolatile semiconductor memory or the like, and stores the system program executed by the CPU 31, various processing programs corresponding to the system program, various application programs, various data and the like. These various program are stored as readable program codes, and the CPU 31 sequentially executes an operation according to the program codes.

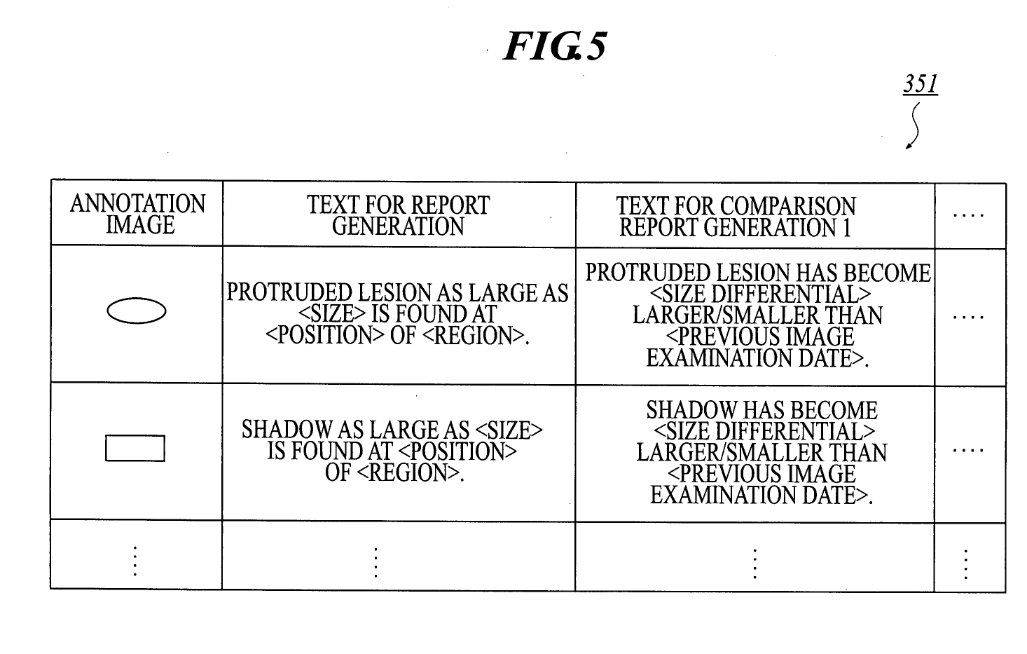

The storing unit 35 comprises an annotation storing unit 351. As shown in FIG. 5, the annotation storing unit 351 stores image data for showing a figure of an annotation (see FIG. 5), letter data indicating text contents for report generation (for example, PROTRUDED LESION AS LARGE AS <SIZE> IS FOUND AT <POSITION> OF <REGION>, SHADOW AS LARGE AS <SIZE> IS FOUND AT <POSITION> OF <REGION>, . . . ), letter data indicating text contents for comparison report generation (for example, PROTRUDED LESION HAS BECOME <SIZE DIFFERENTIAL> LARGER/SMALLER THAN ON <PREVIOUS IMAGE EXAMINATION DATE>, SHADOW HAS BECOME <SIZE DIFFERENTIAL> LARGER/SMALLER THAN ON <PREVIOUS IMAGE EXAMINATION DATE>, . . . ) so as to relate each other. Here, it is assumed that a plurality of types of texts for comparison report generation are stored.

The communication controlling unit 36 comprises a LAN adapter, a router, TA or the like, and controls communications among each apparatus connected to the network N.

Next, an operation of the present embodiment will be described.

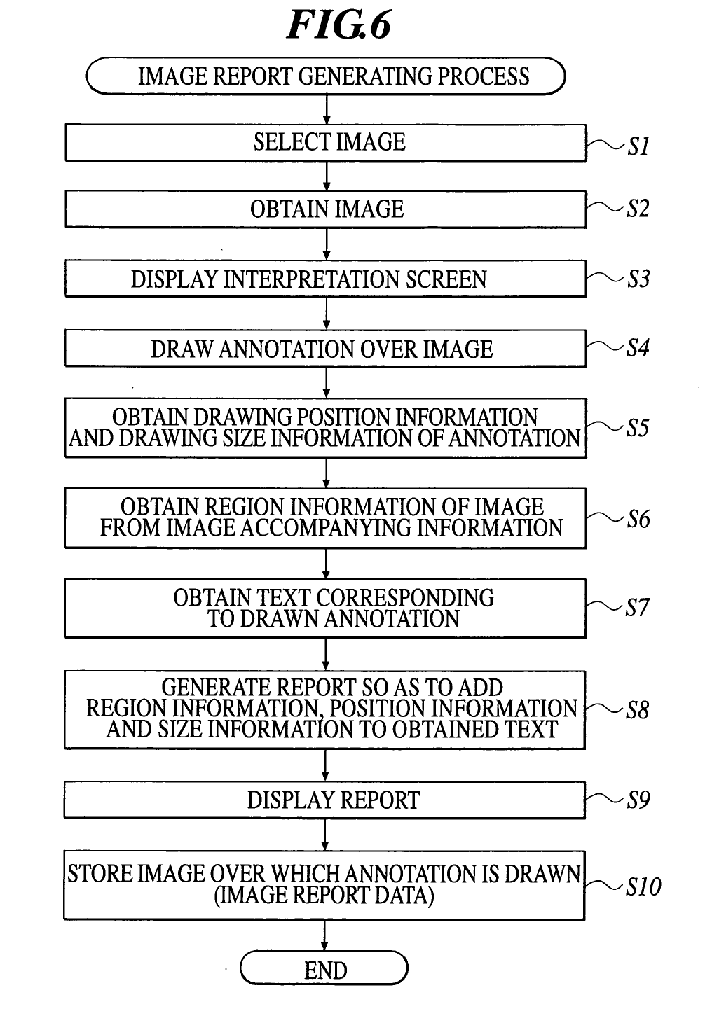

FIG. 6 is a flowchart illustrating an image report generating process executed by the CPU 31 of the interpretation terminal 3. Hereafter, with reference to FIG. 6, the image report generating process will be described.

First, a list of medical images is obtained from the information management apparatus 2 and displayed on the displaying unit 33, and when an image is selected from the list through the operating unit 32 (Step S1), the information management apparatus 2 is requested to deliver image data of the selected image. When the image data is obtained (Step S2), the interpretation screen 331 is displayed on the displaying unit 33, and the obtained image is displayed within an image displaying area of the interpretation screen (Step S3).

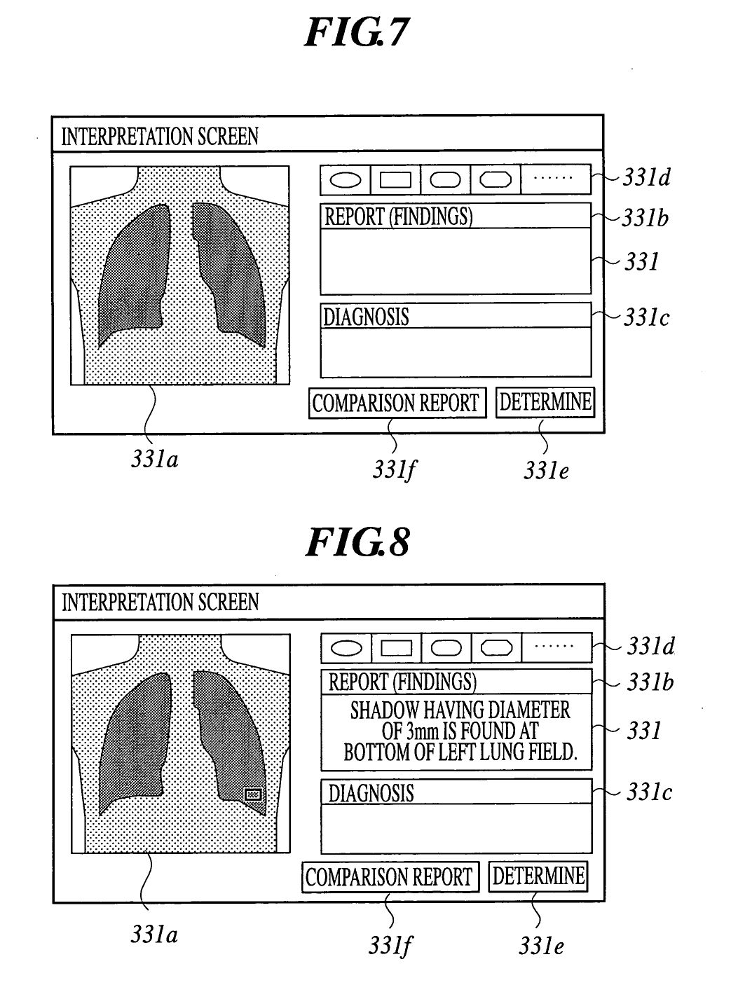

FIG. 7 shows one example of the interpretation screen 331. As shown in FIG. 7, on the interpretation screen 331, displayed are an image display area 331a, a report area 331b and a diagnosis area 331c. Further, above the report area 331b, displayed is an annotation selection area 331d in which a plurality of types of annotations are provided. By selecting a certain annotation from the annotation selection area 331d with a mouse or the like of the operating unit 32, and by designating a certain area in the displayed image with the mouse or the like, it is possible to draw the selected annotation over the image. Here, since it is possible to partially magnify the image displayed on the image display area 331 according to a predetermined operation with the mouse or the like, by designating a certain area in the partially-magnified image with the mouse or the like, it is possible to accurately draw the annotation even in a small area. As shown in FIG. 5, each annotation is related to a text for report generation. Therefore, by selecting an annotation, it is possible to select a text to be written in the report area 331b.

When, on the interpretation screen 331, an annotation is selected through the operating unit 32 and the annotation is drawn over an area of interest in the image display area (Step S4), drawn position information and drawn size information of the annotation are obtained (Step S5), and region information of the image is obtained from accompanying information of the image (Step S6). Further, a text for report generation corresponding to the drawn annotation is obtained from the annotation storing unit 351 (Step S7).

When the drawn position information of the annotation, the drawn size information of the annotation, the region information of the annotation and the text for report generation corresponding to the annotation are obtained, a report is generated so as to add the region information, the drawn position information and the drawn size information to the obtained text (Step S8), and the generated report is displayed on the report area 331b of the interpretation screen 331 (Step S9). When an image diagnosis result by a doctor is inputted in the diagnosis area 331c with a keyboard or the like of the operating unit 32, and a determination button 331e is operated, image report data and the inputted diagnosis information are transmitted to the information management apparatus 2, the image report data being image data to which the annotation is added. Then, the image report data is stored in the image report DB 252 and the diagnosis information is stored in the diagnosis information storing unit 253 (Step S10).

FIG. 8 is a view showing one example of a report generated by the above-described image report generating process, displayed on the report area 331b of the interpretation screen 331.

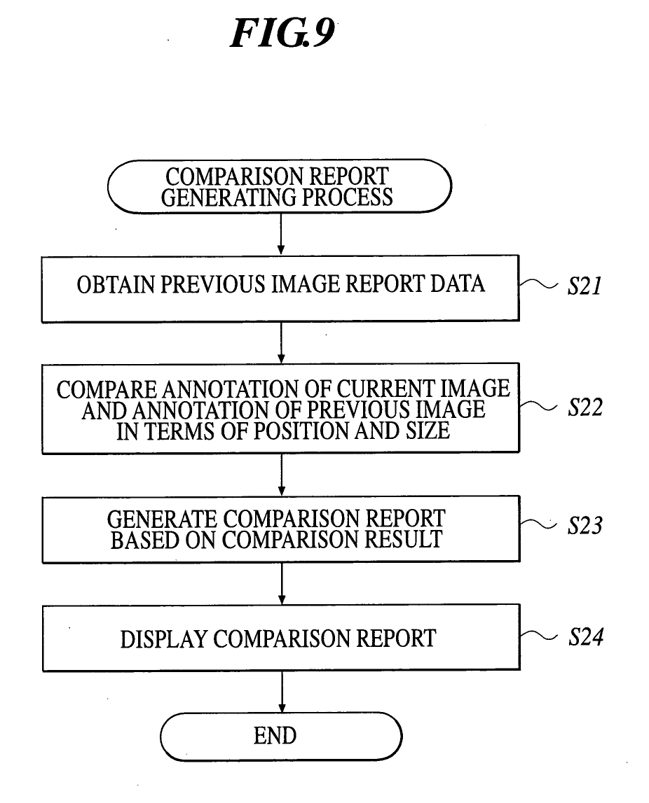

By comparing the image report data generated by the above-described image report generating process with previous image report data, it is possible to generate a comparison report. FIG. 9 is a flowchart illustrating a comparison report generating process which is executed when generation of a comparison report is instructed on the interpretation screen 331 (for example, in the interpretation screen 331 of FIG. 7, when “COMPARISON REPORT” button 331f is operated). By executing the comparison report generating process, the comparison report generating section is realized. Hereinafter, the comparison report generating process will be described with reference to FIG. 9.

First, the information management apparatus 2 is requested to deliver image report data regarding a previous image of the currently-displayed image (an image of the same patient and the same region), and the previous image report data is obtained (Step S21). Next, sizes and positions are compared between an annotation on the image report data of the current image and an annotation on the image report data of the previous image (an identical annotation to the current image) (Step S22). Then, based on the comparison result, a comparison report is generated with reference to the annotation storing unit 351 (Step S23), and the comparison report is displayed on the displaying unit 33 (Step S24).

For example, in the above-described report generating process, based on the comparison between image report data of a current image and that of a previous image generated on 2001/03/01, a comparison report such as “diameter of shadow has grown up at X mm larger than 2001/03/01” or the like is generated and displayed on the displaying unit 33.

As described above, in the interpretation terminal 3, an annotation and a text for report generation are stored so as to relate each other, and when the annotation is drawn over an image, region information of the image, drawn position information of the annotation, drawn size information and a text corresponding to the annotation are obtained, and a report is generated to be displayed on the displaying unit 33.

Accordingly, it is possible to automatically generate a report directly from the appearance of the image, and thereby it is possible to omit the labor of an interpretation doctor to create a text of a report and to input it from a keyboard or the like. Further, since a report is automatically generated based on a text corresponding to a drawn annotation, it is possible to unify expressions of reports into a definite format. Further, it is possible to easily change a format of a text by changing a text corresponding to an annotation. Further, by storing generated image report data in the image report DB 252 so as to relate it with image data of a medical image and its accompanying information, it is possible to utilize them in various ways. For example, by generating a comparison report based on image report data of a current image and that of a previous image, it is possible to provide information which is helpful for diagnosis in comparison with the previous image.

Here, the description of the embodiment above is one example of the medical image interpretation system 100 according to the present invention, and the present invention is not limited to the description.

For example, in the above-described embodiment, image report data is stored as image data. However, a type for identifying drawn position information of an annotation, drawn size information of the annotation and the annotation may be stored.

Further, in the above-described embodiment, the present invention is realized by a structure in which an image DB and an image report DB are provided in the information management apparatus 2, and the information management apparatus 2 and the interpretation terminal 3 are connected through the network N. Alternatively, in the case that a network is not constructed in a hospital or the like, if the image DB and the image report DB is provided in an apparatus which comprises a function of the interpretation terminal 3, it is possible to realize the present invention with a stand-alone apparatus.

And so forth, the detailed structure and the detailed operation of components of the medical image interpretation system in the present embodiment can be suitably changed without departing the gist of the present invention.

The entire disclosure of a Japanese Patent Application No. Tokugan 2003-383587 filed on Nov. 13, 2003, including specifications, claims, drawings and summaries are incorporated herein by reference in their entirety.

1. A medical image interpretation system for displaying a medical image generated from a subject and for generating an interpretation report of the medical image, comprising:
a drawing section for drawing an annotation on an area of interest over the displayed medical image, the annotation being previously related to a text for interpretation report generation; and
a report generating section for obtaining drawn position information and drawn size information of the annotation drawn by the drawing section, for obtaining region information of the medical image, and for generating the interpretation report of the medical image based on the obtained information and the text for interpretation report generation corresponding to the drawn annotation.

2. The system of claim 1, further comprising a storing section for storing image data of the medical image and image report data generated by drawing the annotation so as to relate each other.

3. The system of claim 2, further comprising a comparison report generating section for obtaining image report data of a previous image which is generated according to an identical region of an identical subject to the medical image, and for generating a comparison report by comparing the image report data of the medical image with the image report data of the previous image.

4. An interpretation report generating method in a medical image interpretation system for displaying a medical image generated from a subject and for generating an interpretation report of the medical image, comprising:
drawing an annotation on an area of interest over the displayed medical image, the annotation being previously related to a text for interpretation report generation;
obtaining drawn position information and drawn size information of the drawn annotation;
obtaining region information of the medical image; and
generating the interpretation report of the medical image based on the obtained information and the text for interpretation report generation corresponding to the drawn annotation.
